(19) United States
(12) Patent Application Publication
(10) Pub. No.: US 2010/0191019 A1
(43) Pub. Date: Jul. 29, 2010

(54) Organic Compounds

(76) Inventors: Thierry Granier, Duebendorf (CH); Boris Schilling, Knonau (CH)

Correspondence Address: PARFOMAK, ANDREW N.;NORRIS MCLAUGHLIN & MARCUS PA, 875 THIRD AVE, 8TH FLOOR, NEW YORK, NY, 10022, US

(21) Appl. No.: 12/668,586

(22) PCT Filed: Jul. 15, 2008
(86) PCT No.: PCT/CH08/00316

§ 371 Date: Jan. 11, 2010

(30) Foreign Application Priority Data
Jul. 17, 2007 (GB) 0713860.5

(51) Int. Cl. C07C 49/203; C07C 31/125; C12P 7/26; C12P 7/04; A23L 1/226

(52) U.S. Cl. 568/417; 568/840; 435/148; 435/157

(57) ABSTRACT

The invention refers to conjugated secondary alkatrienol derivatives, a method of their production and to flavour and perfume compositions comprising them.

The present invention refers to conjugated secondary alkatrienol derivatives useful as flavour or fragrance. This invention relates furthermore to a method for their production and to flavour and fragrance compositions comprising them and to fragranced and flavoured applications.

In the flavour and fragrance industry there is a constant demand for new compounds that enhance, modify or improve on organoleptic properties. Surprisingly, a new class of compounds was found, possessing interesting notes, in the direction of green, fruity, undecatriene-like notes.

Thus, the present invention refers in one of its aspects to the use as flavour or fragrance of a compound of formula (I)

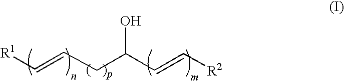

wherein

p is 0 or an integer of 1 to 7, e.g. 2, 3 or 5;

n is 1, 2, or 3; m is 0, 1 or 2; and n+m=3;

R¹ is hydrogen, or linear C₁-C₆ alkyl, e.g. ethyl, n-butyl;

R² is hydrogen, or linear C₁-C₆ alkyl, e.g. ethyl, n-propyl, n-butyl, n-pentyl; and

the total number of carbon atoms of the compound is 7 to 16, e.g. 10, 11 or 12; with the proviso that if m is 0, R² is linear C₁-C₆ alkyl.

All three double bonds are in E configuration or at least one, two or three of them are in Z configuration.

The compounds of formula (I) comprise one chiral centre and as such may exist as a mixture of stereoisomers, or they may be resolved as isomerically pure forms. Resolving stereoisomers adds to the complexity of manufacture and purification of these compounds, and so it is preferred to use the compounds as mixtures of their stereoisomers simply for economic reasons. However, if it is desired to prepare individual stereoisomers, this may be achieved according to methods known in the art, e.g. preparative HPLC and GC, crystallization or stereoselective synthesis.

In particular embodiments compounds of formula (I) are selected from (6E,8E)-undeca-6,8,10-trien-5-ol, (5E,7E,9E)-undeca-5,7,9-trien-4-ol, (5E,7E,9Z)-undeca-5,7,9-trien-4-ol, (6Z,8E)-undeca-6,8,10-trien-5-ol, (6E,8E)-undeca-6,8,10-trien-5-ol, (6E,8E)-undeca-6,8,10-trien-2-ol, (6E,8E)-undeca-6,8,10-trien-3-ol, (4E,6E)-undeca-1,4,6-trien-3-ol, (5E,7E)-dodeca-2,5,7-trien-4-ol and (5E,7E)-dodeca-1,5,7-trien-4-ol, and mixtures thereof.

The compounds according to the present invention may be used alone or in combination with other fragrances or flavours. Preferably however, the compounds of formula (I) may be combined with other fragrances selected from the extensive range of natural and synthetic molecules currently available, such as ethereal oils and extracts, alcohols, aldehydes and ketones, ethers and acetals, esters and lactones, macrocycles and heterocycles. In a further embodiment the compounds of formula (I) may be admixed with one or more ingredients or excipients conventionally used in conjunction with flavours or fragrances in fragranced/flavoured applications, for example, carrier materials, and other auxiliary agents, such as solvents, commonly used in the art.

The following list comprises examples of known fragrances, which may be combined with the compounds of formula (I):
ethereal oils and extracts, e.g. oak moss absolute, basil oil, tropical fruit oils, such as bergamot oil and mandarine oil, mastic absolute, myrtle oil, palmarosa oil, patchouli oil, petitgrain oil, wormwood oil, lavender oil, rose oil, jasmin oil, ylang-ylang oil and sandalwood oil.
alcohols, e.g. cis-3-hexenol, cinnamic alcohol, citronellol, Ebanol®, eugenol, farnesol, geraniol, menthol, nerol, rhodinol, Super Muguet™, linalool, phenylethyl alcohol, Sandalore®, terpineol and Timberol® (1-(2,2,6-Trimethylcyclohexyl)hexan-3-ol).
aldehydes and ketones, e.g. citral, hydroxycitronellal, Lilial®, methylnonylacetaldehyde, anisaldehyde, allylionone, verbenone, nootkatone, geranylacetone, α-amylcinnamic aldehyde, Georgywood™, hydroxycitronellal, Iso E Super®, Isoraldeine® (methylionone), Hedione®, maltol, methyl cedryl ketone, and vanillin.
ethers and acetals, e.g. Ambrox®, geranyl methyl ether, rose oxide or Spirambrene®.
esters and lactones, e.g. benzyl acetate, cedryl actetate, γ-decalactone, Helvetolide®, γ-undecalactone, vetivenyl acetate, cinnamyl propionate, citronellyl acetate, decyl acetate, dimethylbenzylcarbinyl acetate, ethyl acetoacetate, ethyl acetylacetate, cis-3-hexenyl isobutyrate, linalyl acetate and geranyl acetate.
macrocycles, e.g. ambrettolide, ethylene brassylate or Exaltolide®.
heterocycles, e.g. isobutylchinoline.

The compounds of formula (I) may be used in a broad range of fragranced applications, e.g. in any field of fine and functional perfumery, such as perfumes, household products, laundry products, body care products and cosmetics, or in flavoured applications in the field of food, beverages and other edible products. They may be used in fruity and spicy flavour compositions as well as e.g. celery and fishy flavour compositions. In particular they may be used in green vegetable compositions.

The compounds of formula (I) can be employed in widely varying amounts, depending upon the specific application and on the nature of the composition or application one intends to fragrance or flavour, for example the nature and quantity of co-ingredients, and the particular effect that the perfumer or flavourists seeks. In general, the proportion is typically from 0.0001 to 2 weight percent of the application. In one embodiment, compounds of the present invention may be employed in a fabric softener in an amount of from 0.0001 to 0.005 weight percent. In another embodiment, compounds of the present invention may be used in an alcoholic solution in amounts of from 0.01 to 3 weight percent, more preferably between 0.5 and 2 weight percent. However, these values are given only by way of example, since the experienced perfumer and flavourist may also achieve effects or may create novel accords with lower or higher concentrations, e.g. up to about 20 weight percent based on the composition.

The compounds of formula (I) may be employed into a consumer product base by mixing a compound of formula (I), a mixture thereof or a flavour or fragrance composition comprising it, with the consumer product base, and/or they may, in an earlier step, be entrapped with an entrapment material such as polymers, capsules, microcapsules and nanocapsules, liposomes, film formers, absorbents such as carbon or zeolites, cyclic oligosaccharides and mixtures thereof, and/or they may be chemically bonded to substrates, which together with the substrate forms a precursor, which are adapted to release the compound of formula (I) upon application of an external stimulus such as light, enzyme, or the like, and then mixed with the consumer product base.

The invention additionally provides a method of manufacturing a flavour or fragrance application comprising the incorporation of a compound of formula (I) as flavour or fragrance ingredient, either by admixing the compound to the consumer product base or by admixing a composition comprising a compound of formula (I) or a precursor thereof, which may then be mixed to the consumer product base, using conventional techniques and methods. Through the addition of an organoleptically acceptable amount of a compound of formula (I) or a mixture thereof, the organoleptic properties of the consumer product base will be improved, enhanced or modified.

By “precursors” is meant, reaction products of a compound of formula (I) with a compound comprising at least one functional group selected from the group of primary amine, secondary amine, sulphydryl (thiol), hydroxyl and carboxyl, in which a covalent bond is formed between at least one carbon atom of the compound of formula (I) and at least one of the hetero atoms of said compounds comprising at least one functional group selected from the group of N, S and O.

The invention furthermore provides a method for improving, enhancing or modifying a consumer product base through the addition thereto of a compound of formula (I), or a mixture thereof.

The invention also provides a flavour or fragrance application comprising:
a) as flavour or fragrance, a compound of formula (I) or a mixture thereof; and
b) a consumer product base.

When used as fragrance, by “consumer product base” is meant a formulation for use as a consumer product to fulfill specific actions, such as cleaning, softening, and caring or the like. Examples of such products include fine perfumery, e.g. perfume and eau de toilette; fabric care, household products and personal care products such as laundry care detergents, rinse conditioner, personal cleansing products, detergent for dishwasher, surface cleaner; laundry products, e.g. softener, bleach, detergent; body care products, e.g. shampoo, shower gel; air care products and cosmetics, e.g. deodorant, and vanishing crème. This list of products is given by way of illustration and is not to be regarded as being in any way limiting.

When used as flavour, by “consumer product base” is meant the base formulation of an edible product, for example processed food, beverages or chewable articles such as chewing gum. It refers to the base formulation of the edible product, without the compound of formula (I).

Compounds of formula (I) are obtainable, for example, from the appropriate unsaturated aldehyde using classical organic chemistry procedures known to the person skilled in the art. The compounds of formula (I) may also be prepared by reduction of the appropriate ketone under conditions known in the art.

Optionally the compounds of formula (I) are obtainable by enzymatic oxidation, comprising contacting a triene of formula (II)

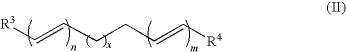

wherein
x is 0 or an integer of 1 to 7, e.g. 2, 3 or 5;
n is 1, 2, or 3; m is 0, 1 or 2; and n+m=3;
R³ is hydrogen, or linear C₁-C₆ alkyl, e.g. ethyl, n-butyl;
R⁴ is hydrogen, or linear C₁-C₆ alkyl, e.g. ethyl, n-propyl, n-butyl, n-pentyl; and
the total number of carbon atoms of the compound is 7 to 16, e.g. 10, 11 or 12;
with the proviso that if m is 0, R⁴ is linear C₁-C₆ alkyl;
   with a cytochrome P450 enzyme.

Cytochrome P450 (CYP) enzymes constitute a sub-family of heme-thiolate enzymes, which catalyse primarily mono-oxygenase reactions involving a two-stage reduction of molecular oxygen and subsequent single-oxygen atom insertion, although reductive metabolism is also known. CYP2E1 is a major isoform expressed in the human liver but also other parts of the human body. CYP2E1 metabolizes hydrophilic substrates such as ethanol and acetone, small molecules such as benzene and chloroform, but also fatty acids. CYP-catalyzed reactions are diverse and hydroxylation, epoxidation of double-bonds, but also demethylation of C-methyl and N-methyl group occur. While investigating the specificity of CYP enzymes with respect to metabolism of odorant molecules, applicants identified that CYP2E1 is metabolizing a triene of formula (II) to produce different oxidized species.

In one embodiment the compound of formula (II) is undecatriene, which may be synthetically prepared or one may use an essential oil comprising it, e.g. 1,3,5-undecatriene which is present in minor amounts in Galbanum oil.

The invention is now further described with reference to the following non-limiting examples. These examples are for the purpose of illustration only and it is understood that variations and modifications can be made by one skilled in the art.

### EXAMPLE 1

### (6E,8E)-undeca-6,8,10-trien-5-one

Synthesis of the starting materials. Dimethyl (2-oxohexyl)phosphonate was prepared (33-41% yield) by acylation of dimethyl methylphosphonate with methyl pentanoate using LDA (lithium diisopropylamide) as a base according to a literature procedure (Aboujaoude, E., E.; Collignon, N.; Teulade, M.-P.; Savignac, P. Phosphorus and Sulfur 1985, 25, 57-61). Pentadienal was prepared from pentadienoic acid according to a literature procedure (Schneider, M. P.; Goldbach, M. J. Am. Chem. Soc. 1980, 102, 6114-6) by LiAlH₄-reduction (36-66% yield) and subsequent PCC-oxidation in the presence of sodium acetate (17-46% yield).

A suspension of sodium hydride (0.9 g, 60% suspension in oil, 23 mmol) in dimethoxyethane (50 ml) was treated within 20 min. with a solution of dimethyl (2-oxohexyl)phosphonate (4.7 g, 22.7 mmol) in dimethoxyethane (50 ml) while the reaction temperature rose to 30° C. The resulting mixture was diluted with dimethoxyethane (20 ml) and stirred for 15 min. at 25° C. A solution of pentadienal (1.33 g, 16 mmol) in dimethoxyethane (30 ml) was then added dropwise during 20 min. and the resulting mixture was stirred 90 min. at 25° C. before being added to an ice-cold solution of 2M aqueous HCl (150 ml). The mixture was extracted twice with methyl tert.-butyl ether (100 ml) and the combined organic phases were washed twice with saturated aqueous NaCl solution, dried (MgSO₄), filtered, and concentrated. FC (hexane/methyl tert.-butyl ether 18:1) of the crude product gave (6E,8E)-undeca-6,8,10-trien-5-one (1.52 g, 57%). Boiling point: 110° C. (0.1 mbar).

¹H-NMR (400 MHz, CDCl₃): δ 7.18 (dd, J=11.0, 15.4, H—C (7)), 6.60 (dd, J=10.8, 14.9, H—C (9)), 6.44 (dt, J=10.4, 17.0, H—C(10)), 6.33 (dd, J=11.1, 14.7, H—C (8)), 6.19 (d, J=15.5, H—C (6)), 5.43 (br. d, J=16.8, H—C (11)), 5.33 (br. d, J=9.9, H—C (11)), 2.56 (t, J=7.5, 2H—C (4)), 1.61 (quint., J=7.5, 2H—C(3)), 1.35 (sext., J=7.4, 2H—C (2)), 0.92 (t, J=7.3, MeC (2)).

¹³C-NMR (100 MHz, CDCl₃): δ 200.69 (s, CO), 141.79 (d), 141.50 (d), 136.20 (d), 130.94 (d), 129.86 (d), 121.68 (t, C(11)), 40.46 (t, C(4)), 26.49 (t, C(3)), 22.40 (t, C(2)), 13.86 (q, C(1)).

MS (EI): 164 (10), 149 (3), 146 (1), 145 (1), 137 (2), 135 (3), 131 (2), 122 (28), 121 (10), 108 (8), 107 (88), 94 (6), 80 (14), 79 (100), 78 (17), 77 (82), 65 (3), 57 (9), 55 (7), 54 (1), 53 (15), 52 (8), 51 (15), 41 (11), 39 (13), 29 (8), 27 (10).

IR: vₘₐₓ 2953, 2933, 2868, 1679, 1618, 1599, 1574, 1415, 1377, 1344, 1312, 1291, 1259, 1227, 1125, 1054, 1012, 907, 891 cm⁻¹.

UV (CHCl₃): λ (log ε) 280 (4.1), 230 (3.8).

Odour description: floral, green, metallic.

### EXAMPLE 2

### (6E,8E)-undeca-6,8,10-trien-5-ol

A mixture of (6E,8E)-undeca-6,8,10-trien-5-one (1.5 g, 9.1 mmol) and anhydrous cerium trichloride (2.25 g, 9.1 mmol) in methanol (25 ml) was stirred at 20° C. for 30 min. and added within 30 min. to a suspension of sodium borohydride (0.24 g, 6.4 mmol) in methanol (20 ml) at 5° C. The resulting mixture was then stirred 3 h at 20° C. and added to an ice-cold solution of saturated aqueous NH₄Cl (50 ml). The mixture was extracted twice with methyl tert.-butyl ether (80 ml) and the combined organic phases were washed with saturated aqueous NaCl solution (80 ml), dried (MgSO₄), filtered, and concentrated. FC (hexane/methyl tert.-butyl ether 5:1) of the crude product (1.3 g) gave (6E,8E)-undeca-6,8,10-trien-5-ol (1.0 g, 66%). Boiling point: 100° C. (0.1 mbar).

¹H-NMR (400 MHz, CDCl₃): δ 6.42-6.31 (m, 1H), 6.28-6.20 (m, 3H), 5.78-5.69 (m, 1H, H—C (6)), 5.23 (dd, J=1.6, 16.5, H—C (11)), 5.10 (dd, J=1.6, 9.7, H—C (11)), 4.15 (br. q, J=6.7, H—C (5)), 1.62 (br. s, OH), 1.63-1.47 (m, 2H), 1.41-1.25 (m, 4H), 0.90 (t, J=7.1, MeC(2)).

¹³C-NMR (100 MHz, CDCl₃): δ 136.93 (d), 136.80 (d), 133.46 (d), 132.40 (d), 130.18 (d), 117.50 (t, C(11)), 72.67 (d, C(5)), 36.97 (t, C(4)), 27.53 (t, C(3)), 22.60 (t, C(2)), 14.00 (q, C(1)).

MS (EI): 166 (11), 151 (1), 150 (3), 149 (6), 148 (52), 137 (2), 133 (3), 119 (25), 117 (11), 115 (7), 109 (35), 107 (8), 106 (12), 105 (67), 104 (6), 103 (10), 93 (9), 92 (21), 91 (99), 85 (75), 81 (42), 80 (15), 79 (67), 78 (23), 77 (36), 67 (31), 66 (12), 65 (21), 57 (74), 55 (28), 53 (27), 51 (18), 41 (100), 39 (42), 29 (26), 27 (27).

IR: vₘₐₓ 3335, 2957, 2930, 2860, 1626, 1466, 1417, 1379, 1284, 1169, 1128, 1045, 1003, 972, 897 cm⁻¹.

UV (CHCl₃): λ (log ε) 277 (4.4), 266 (4.5), 257 (4.4), 225 (4.0).

Odour description: green, floral, metallic.

### EXAMPLE 3

### (5E,7E,9E)-undeca-5,7,9-trien-4-one and (5E,7E,9Z)-undeca-5,7,9-trien-4-one

Dimethyl (2-oxopentyl)phosphonate was prepared in 29% yield by acylation of dimethyl methylphosphonate with ethyl butanoate using LDA as a base according to a literature procedure (Aboujaoude, E., E.; Collignon, N.; Teulade, M.-P.; Savignac, P. Phosphorus and Sulfur 1985, 25, 57-61).

A suspension of sodium hydride (5.9 g, 60% suspension in oil, 148 mmol) in dimethoxyethane (70 ml) was treated within 40 min. with a solution of dimethyl (2-oxopentyl)phosphonate (28.8 g, 148 mmol) in dimethoxyethane (80 ml) while the reaction temperature rose to 33° C. and the resulting mixture was stirred for 20 min. at 35° C. At 45° C., a solution of trans,trans-2,4-hexadienal (11.3 g, 106 mmol, 85:15 mixture of 2E4E/2E4Z-isomers) in dimethoxyethane (50 ml) was then added dropwise during 60 min. and the resulting mixture was stirred 75 min. at 50° C. before being cooled down and added to an ice-cold solution of saturated aqueous NH₄Cl (200 ml). The mixture was extracted three times with diethyl ether (150 ml) and the combined organic phases were washed with saturated aqueous NaCl solution (100 ml), dried (MgSO₄), filtered, and concentrated. The crude product (24.5 g) was distilled using a Kugehlrohr apparatus (ball-to-ball rotatory distillation apparatus) at 130° C. and 0.1 mbar. FC (hexane/MTBE 16:1) of the main fraction (12.6 g) gave a 84:16 mixture of (5E,7E,9E/Z)-undeca-5,7,9-trien-4-one (9.5 g, 54%). Boiling point: 95° C. (0.09 mbar).

IR: vₘₐₓ 3019, 2962, 2875, 1682, 1658, 1600, 1578, 1447, 1410, 1367, 1261, 1189, 1125, 1041, 1000, 926, 890 cm⁻¹.

UV (CHCl₃): λ (log ε) 316 (4.5), 229 (3.7).

Odour description: citrus, fresh, aldehydic, allylic, metallic, fruity

Data of (5E,7E,9E)-undeca-5,7,9-trien-4-one:

¹H-NMR (400 MHz, CDCl₃): δ 7.18 (ddd, J=0.5, 11.2, 15.4, H—C (6)), 6.57 (ddquint., J=0.6, 10.6, 14.9, H—C (8)), 6.20 (ddquint., J=0.8, 11.1, 14.9, H—C (7)), 6.21-6.13 (m, 1H, H—C (9)), 6.14 (br. d, J=15.4, H—C (5)), 5.95 (br. dq, J=7.0, 14.4, H—C (10)), 2.53 (t, J=7.3, 2H—C (3)), 1.83 (dd, J=1.2, 6.8, MeC(10)), 1.65 (sext., J=7.4, 2H—C (2)), 0.94 (t, J=7.4, MeC(2)).

¹³C-NMR (100 MHz, CDCl₃): δ 200.64 (s, CO), 142.63 (d), 141.79 (d), 135.27 (d), 131.31 (d), 128.63 (d), 128.09 (d), 42.52 (t, C(4)), 18.55 (q, C(11)), 17.89 (t, C(2)), 13.82 (q, C(1)).

MS (EI): 164 (37), 149 (5), 136 (3), 135 (18), 131 (2), 122 (9), 121 (100), 107 (5), 105 (3), 93 (56), 91 (69), 79 (12), 78 (18), 77 (72), 71 (11), 65 (19), 55 (10), 53 (12), 52 (6), 51 (12), 43 (23), 41 (24), 39 (24), 27 (11).

Data of (5E,7E,9Z)-undeca-5,7,9-trien-4-one:

¹H-NMR (400 MHz, CDCl₃): δ 7.24 (ddd, J=0.7, 11.3, 15.5, H—C (6)), 6.92 (ddquint., J=0.8, 11.4, 15.0, H—C (8)), 6.30 (ddquint., J=0.7, 11.3, 14.8, H—C (7)), 6.22-6.10 (m, 2H, H—C (9), H—C (5)), 5.77 (br. dq, J=7.3, 10.6, H—C (10)), 2.54 (t, J=7.3, 2H—C (3)), 1.84 (d, J=7.3, MeC(10)), 1.66 (sext., J=7.3, 2H—C (2)), 0.95 (t, J=7.3, MeC(2)).

¹³C-NMR (100 MHz, CDCl₃): δ 200.64 (s, CO), 142.52 (d), 136.33 (d), 131.81 (d), 130.07 (d), 129.07 (d), 128.94 (d), 42.52 (t, C(4)), 18.55 (q, C(11)), 17.87 (t, C(2)), 13.80 (q, C(1)).

MS (EI): 164 (36), 149 (5), 136 (3), 135 (18), 131 (2), 122 (9), 121 (100), 107 (5), 105 (3), 93 (58), 91 (71), 79 (13), 78 (19), 77 (75), 71 (11), 65 (19), 55 (11), 53 (12), 52 (6), 51 (12), 43 (24), 41 (25), 39 (26), 27 (12).

### EXAMPLE 4

### (5E,7E,9E)-undeca-5,7,9-trien-4-ol and (5E,7E,9Z)-undeca-5,7,9-trien-4-ol

A 84:16 mixture of (5E,7E,9E/Z)-undeca-5,7,9-trien-4-one (3.5 g, 21.3 mmol) and anhydrous cerium trichloride (5.25 g, 21.3 mmol) in methanol (50 ml) was stirred at 20° C. for 45 min. and added within 45 min. to a suspension of sodium borohydride (0.56 g, 14.9 mmol) in methanol (30 ml) at 5° C. The resulting mixture was then stirred 3 h at 20° C. and added to an ice-cold solution of saturated aqueous NH₄Cl (50 ml). The mixture was extracted twice with methyl tert.-butyl ether (100 ml) and the combined organic phases were washed with saturated aqueous NaCl solution (100 ml), dried (MgSO₄), filtered, and concentrated. FC (hexane/methyl tert.-butyl ether 6:1) of the crude product (3.6 g) gave a 84:16 mixture of (5E,7E,9E/Z)-undeca-5,7,9-trien-4-ol (2.3 g, 65%). Boiling point: 125° C. (0.13 mbar).

IR: vₘₐₓ 3349, 3016, 2958, 2931, 2873, 1637, 1449, 1378, 1126, 1069, 991, 927, 847 cm⁻¹. UV (CHCl₃): λ (log ε) 281 (4.5), 271 (4.6), 260 (4.5), 225 (4.0).

Odour description: fruity, fatty, floral

Data of (5E,7E,9E)-undeca-5,7,9-trien-4-ol:

¹H-NMR (400 MHz, CDCl₃): δ 6.32-6.01 (m, 4H), 5.72 (dq, J=6.8, 15.4, H—C (10)), 5.65 (dd, J=7.1, 15.2, H—C (5)), 4.14 (br. q, J=6.7, H—C (4)), 1.78 (dd, J=1.3, 6.8, MeC(10)), 1.63 (br. s, OH), 1.61-1.29 (m, 4H), 0.92 (t, J=7.2, MeC(2)).

¹³C-NMR (100 MHz, CDCl₃): δ 135.32 (d), 133.29 (d), 131.49 (d), 130.71 (d), 130.20 (d), 129.39 (d), 72.55 (d, C(4)), 39.41 (t, C(3)), 18.62 (t, C(2)), 18.28 (q, C(11)), 13.96 (q,))

MS (EI): 166 (61), 151 (2), 150 (2), 149 (8), 148 (75), 137 (2), 133 (13), 123 (49), 119 (55), 117 (12), 115 (7), 105 (53), 95 (41), 94 (7), 93 (33), 92 (16), 91 (100), 81 (41), 80 (23), 79 (56), 78 (16), 77 (42), 71 (91), 67 (30), 66 (1), 65 (15), 55 (40), 53 (14), 43 (42), 41 (42), 39 (21), 29 (11), 27 (16).

Data of (5E,7E,9Z)-undeca-5,7,9-trien-4-ol:

¹H-NMR (400 MHz, CDCl₃): selected signals 86.52 (dd, J=11.1, 14.7, 1H), 5.53 (br. dq, J=7.2, 10.7, H—C (10)), 4.16 (br. q, J=7.0, H—C (4)), 0.93 (t, J=7.2, MeC(2)).

¹³C-NMR (100 MHz, CDCl₃): δ 135.99 (d), 131.44 (d), 130.74 (d), 129.24 (d), 128.17 (d), 127.12 (d), 72.49 (d, C(4)), 39.41 (t, C(3)), 18.62 (t, C(2)), 18.28 (q, C(11)), 13.45 (q, C(1))

MS (EI): 166 (46), 151 (2), 150 (2), 149 (7), 148 (53), 137 (2), 133 (11), 123 (40), 119 (44), 117 (11), 115 (6), 105 (42), 95 (41), 94 (8), 93 (29), 92 (15), 91 (89), 81 (40), 80 (21), 79 (48), 78 (15), 77 (39), 71 (100), 67 (25), 66 (6), 65 (15), 55 (39), 53 (15), 43 (46), 41 (39), 39 (22), 29 (12), 27 (15).

### EXAMPLE 5

### (6Z,8E)-undeca-6,8,10-trien-5-ol and (6E,8E)-undeca-6,8,10-trien-5-ol

Synthesis of the starting materials. bis(2,2,2-Trifluoroethyl) (2-oxohexyl)phosphonate was prepared in 53% yield by acylation of bis(2,2,2-trifluoroethyl)methylphosphonate with pentanoyl chloride using lithium bis(trimethylsilyl)amide (1M in tetrahydrofuran) as a base according to a literature procedure (Yu, W.; Su, M.; Jin, Z. Tetrahedron Letters 1999, 40, 6725-8).

At −50° C., a solution of bis(2,2,2-trifluoroethyl) (2-oxohexyl)phosphonate (18.0 g, 52 mmol) and 18-crown-6 (16.6 g, 62.9 mmol) in tetrahydrofuran (200 ml) was treated with potassium bis(trimethylsilyl)amide (10.4 g, 95%, 49.4 mmol) and stirred 1 h. Pentadienal (4.0 g, 48.4 mmol, prepared as in example 1) was then added within 10 min. and the resulting mixture was stirred for 1 h before being poured into an ice-cold solution of saturated aqueous NH₄Cl (200 ml). The mixture was extracted three times with methyl tert.-butyl ether (100 ml) and the combined organic phases were washed with saturated aqueous NaCl solution (100 ml), dried (MgSO₄), filtered, and concentrated. The crude product (17.3 g (6Z,8E)-undeca-6,8,10-trien-5-one and (6E,8E)-undeca-6,8,10-trien-5-one, 6Z/6E 73:27) was dissolved in methanol (100 ml), stirred 45 min. at 20° C. in the presence of anhydrous cerium trichloride (11.9 g, 48.4 mmol) and the resulting solution was added within 40 min. to a suspension of sodium borohydride (1.83 g, 48.4 mmol) in methanol (80 ml) at 5° C. The resulting mixture was then stirred 3 h at 20° C., treated again with sodium borohydride (0.5 g, 13.2 mmol) and stirred 2 h at 20° C. before being poured into an ice-cold solution of saturated aqueous NH₄Cl (200 ml). The mixture was treated with 2M hydrochloric acid (20 ml) and extracted three times with methyl tert.-butyl ether (100 ml) and the combined organic phases were washed twice with aqueous NaCl solution (100 ml), dried (MgSO₄), filtered, and concentrated. FC (hexane/methyl tert.-butyl ether 6:1) of the crude product gave a 88:12 mixture of (6Z/E,8E)-undeca-6,8,10-trien-5-ol (1.6 g, 20%). Boiling point: 100° C. (0.08 mbar).

Odour description: Top notes: green, violet, pineapple, Undecavertol, Folione.

Dry down notes: more Galbanone-like than Undecatriene-like, Cetone V aspect Data of (6Z,8E)-undeca-6,8,10-trien-5-ol:

¹H-NMR (400 MHz, CDCl₃): δ 6.51 (br. dd, J=11.5, 14.7, H—C (8)), 6.40 (br. dd, J=10.4, 17.0, H—C (10)), 6.25 (ddquint., J=0.7, 10.6, 14.8, H—C (9)), 6.08 (br. tt, J=0.8, 11.2, H—C (7)), 5.44 (br. t, J=10.0, H—C (6)), 5.26 (br. d, J=16.9, H—C (11)), 5.15 (br. d, J=10.2, H—C (11)), 4.58 (br. q, J=7.3, H—C (5)), 1.59 (br. s, OH), 1.70-1.55 (m, 1H), 1.55-1.42 (m, 1H), 1.40-1.25 (m, 4H), 0.90 (t, J=7.0, MeC(2)).

¹³C-NMR (100 MHz, CDCl₃): δ 136.77 (d), 135.03 (d), 134.54 (d), 129.80 (d), 127.80 (d), 118.25 (t, C(11)), 67.99 (d, C(5)), 37.14 (t, C(4)), 27.45 (t, C(3)), 22.60 (t, C(2)), 14.00 (q, C(1)).

MS (EI): 166 (9), 148 (3), 137 (2), 119 (2), 109 (43), 105 (8), 95 (8), 91 (27), 85 (100), 81 (80), 79 (65), 77 (29), 67 (35), 57 (86), 55 (23), 53 (22), 41 (39), 39 (16), 29 (12), 27 (9).

IR: vₘₐₓ 3327, 3019, 2956, 2929, 2859, 1624, 1465, 1378, 1285, 1046, 1002, 938, 897 cm⁻¹.

UV (CHCl₃): λ (log ε) 277 (4.5), 266 (4.6), 257 (4.5), 225 (4.0).

Data of (6E,8E)-undeca-6,8,10-trien-5-ol: see example 2.

### EXAMPLE 6

### (6E,8E)-undeca-6,8,10-trien-2-ol

Synthesis of the starting materials. 2,4-Pentadienyltriphenylphosphonium bromide was prepared from 2,4-pentadienyl bromide and triphenylphosphine as described in the literature (Näf, F.; Decorzant, R.; Thommen, W.; Whillhalm, B.; Ohloff, G. Helv. Chim. Acta 1975, 58, 1016-37).

Tetrahydro-6-methyl-2H-pyran-2-ol. At −73° C., a solution of delta-hexanolactone (20.4 g, 98%, 175 mmol) in dichloromethane (400 ml) was treated within 60 min. with a 1M solution of diisobutylaluminium hydride in hexane (246 ml, 245 mmol). The resulting solution was stirred 4 h at −73° C. before being diluted with methyl tert.-butyl ether (200 ml) and poured into an ice-cold solution of saturated aqueous NH₄Cl (300 ml). The mixture was filtered and the aqueous phase extracted with methyl tert.-butyl ether (150 ml). The combined organic phases were washed with an aqueous solution of NaCl, dried (MgSO₄), filtered, and concentrated to give crude tetrahydro-6-methyl-2H-pyran-2-ol (12.8 g, 62%) that was used as such in the next step.

Synthesis of the trienol. At 5° C., a solution of 2,4-pentadienyltriphenylphosphonium bromide (42.3 g, 103 mmol) in tetrahydrofuran (300 ml) was treated with a 1.6M solution of n-butyl lithium in hexane (67.8 ml, 108 mmol) and stirred 0.5 h. The ice bath was then removed and a solution of tetrahydro-6-methyl-2H-pyran-2-ol (6.0 g, 51.6 mmol) in tetrahydrofuran (80 ml) was added within 20 min. The resulting mixture was refluxed for 24 h before being poured into an ice-cold solution of saturated aqueous NH₄Cl (150 ml). The mixture was extracted twice with methyl tert.-butyl ether (100 ml) and the combined organic phases were washed with saturated aqueous NaCl solution (100 ml), dried (MgSO₄), filtered, and concentrated. FC (hexane/methyl tert.-butyl ether 6:1) of the crude product (26.8 g, 60:40 6E/Z mixture) gave a 1:1 mixture of (6E/Z,8E)-undeca-6,8,10-trien-2-ol (1.4 g, 16%). Boiling point: 99° C. (0.09 mbar).

IR: vₘₐₓ 3334, 3087, 3018, 2964, 2928, 2860, 1624, 1454, 1373, 1312, 1128, 1084, 1003, 973, 938, 895 cm⁻¹.

UV (CHCl₃): λ (log ε) 274 (4.4), 264 (4.5), 257 (4.4).

Odour description: Undecatriene-like, fatty, green, fruity, metallic

Data of (6E,8E)-undeca-6,8,10-trien-2-ol:

¹H-NMR (400 MHz, CDCl₃): δ 6.35 (dt, J=10.0, 16.9, H—C (10)), 6.21 (dd, J=9.9, 14.7, H—C (8)), 6.16 (dd, J=10.0, 16.3, H—C (9)), 6.09 (dt, J=10.3, 15.0, H—C (7)), 5.72 (br. dt, J=7.1, 15.1, H—C (6)), 5.17 (m, H—C (11)), 5.04 (dd, J=1.3, 10.0, H—C (11)), 3.85-3.75 (m, H—C (2)), 2.31-2.08 (m, 2H—C (5)), 1.66-1.36 (m, 2H—C (3)), 2H—C (4), OH), 1.19 (t, J=6.1, MeC(2)).

¹³C-NMR (100 MHz, CDCl₃): δ 137.10 (d), 135.43 (d), 133.17 (d), 131.21 (d), 130.47 (d), 116.35 (t, C(11)), 67.96 (d, C(2)), 38.74 (t, C(5)), 32.67 (t), 25.36 (t), 23.51 (q, C(1)).

MS (EI): 166 (8), 151 (2), 148 (2), 137 (2), 133 (3), 123 (13), 119 (5), 117 (2), 108 (16), 106 (32), 105 (25), 97 (8), 93 (19), 92 (14), 91 (100), 84 (8), 81 (14), 80 (14), 79 (58), 78 (42), 77 (46), 71 (10), 68 (8), 67 (22), 66 (8), 65 (16), 55 (10), 54 (7), 53 (10), 51 (7), 45 (23), 43 (25), 41 (22), 39 (15), 29 (5), 27 (6).

Data of (6Z,8E)-undeca-6,8,10-trien-2-ol:

¹H-NMR (400 MHz, CDCl₃): δ 6.49 (br. dd, J=11.2, 14.9, H—C (8)), 6.40 (dt, J=10.4, 16.8, H—C (10)), 6.21 (br. dd, J=10.7, 14.9, H—C (9)), 6.04 (br. t, J=11.0, H—C (7)), 5.47 (br. dt, J=7.7, 10.7, H—C (6)), 5.21 (br. d, J=16.7, H—C (11)), 5.09 (br. d, J=10.2, H—C(11)), 3.67-3.57 (m, H—C (2)), 2.31-2.08 (m, 2H—C (5)), 1.66-1.36 (m, 2H—C (3)), 2H—C(4), OH), 1.19 (t, J=6.2, MeC(2)).

¹³C-NMR (100 MHz, CDCl₃): δ 137.15 (d), 133.41 (d), 132.89 (d), 128.67 (d), 128.47 (d), 117.02 (t, C(11)), 67.96 (d, C(2)), 38.78 (t, C(5)), 27.74 (t), 25.74 (t), 23.52 (q, C(1)).

MS (EI): 166 (6), 151 (2), 148 (2), 137 (2), 133 (4), 123 (13), 119 (6), 117 (2), 108 (17), 106 (29), 105 (25), 97 (9), 93 (21), 92 (14), 91 (100), 84 (9), 81 (16), 80 (16), 79 (66), 78 (42), 77 (47), 71 (11), 68 (10), 67 (24), 66 (9), 65 (17), 55 (11), 54 (8), 53 (10), 51 (8), 45 (25), 43 (29), 41 (23), 39 (17), 29 (5), 27 (7).

### EXAMPLE 7

### (6E,8E)-undeca-6,8,10-trien-3-ol

### Synthesis of the Starting Materials:

2,4-Pentadienyltriphenylphosphonium bromide. Prepared as described in example 6. 5-Ethyltetrahydro-2-furanol. At −75° C., a solution of gamma-caprolactone (10.2 g, 98%, 87.6 mmol) in dichloromethane (200 ml) was treated within 60 min. with a 1M solution of diisobutylaluminium hydride in hexane (123 ml, 123 mmol). The resulting solution was stirred 3.5 h at −70° C. before being poured into an ice-cold solution of saturated aqueous NH₄Cl (200 ml). The mixture was acidified with a 2M aqueous solution of hydrochloric acid (50 ml) and the organic phase was washed twice with water (100 ml). The aqueous phases were extracted with dichloromethane (100 ml) and the combined organic phases were dried (MgSO₄), filtered, and concentrated to give crude 5-ethyltetrahydro-2-furanol (5.47 g, 53%) that was used as such in the next step.

### Synthesis of the Trienol:

At 5° C., a solution of 2,4-pentadienyltriphenylphosphonium bromide (30.3 g, 74 mmol) in tetrahydrofuran (250 ml) was treated with a 1.6M solution of n-butyl lithium in hexane (48.6 ml, 78 mmol) and stirred 0.5 h. The ice bath was then removed and a solution of 5-ethyltetrahydro-2-furanol (4.3 g, 37.0 mmol) in tetrahydrofuran (50 ml) was added within 15 min. The resulting mixture was refluxed for 3 h before being poured into an ice-cold solution of saturated aqueous NH₄Cl (150 ml). The mixture was extracted twice with methyl tert.-butyl ether (150 ml) and the combined organic phases were washed with saturated aqueous NaCl solution (150 ml), dried (MgSO₄), filtered, and concentrated. FC (hexane/methyl tert.-butyl ether 6:1) of the crude product (20.4 g) gave (6Z,8E)-undeca-6,8,10-trien-2-ol (0.10 g, 1.6%) and a 1:4 mixture of (6Z,8E)- and (6E,8E)-undeca-6,8,10-trien-2-ol (2.15 g, 35%).

Data of the 1:4 mixture of (6Z,8E)- and (6E,8E)-undeca-6,8,10-trien-2-ol:

Boiling point: 95° C. (0.08 mbar).

IR: vₘₐₓ 3331, 3087, 3020, 2962, 2928, 2876, 1625, 1583, 1455, 1187, 1162, 1118, 1003, 971, 930, 894 cm⁻¹.

UV (CHCl₃): λ (log ε) 273 (4.6), 263 (4.7), 255 (4.5).

Odour description: green, Undecatriene-like, floral, fruity, woody, leather

Data of (6E,8E)-undeca-6,8,10-trien-3-ol:

¹H-NMR (400 MHz, CDCl₃): δ 6.34 (dt, J=10.0, 16.9, H—C (10)), 6.20 (dd, J=9.9, 14.5, H—C (8)), 6.17 (dd, J=9.7, 15.4, H—C (9)), 6.12 (dt, J=10.2, 15.1, H—C (7)), 5.74 (br. dt, J=7.1, 15.1, H—C (6)), 5.17 (dd, J=1.3, 16.8, H—C (11)), 5.05 (dd, J=1.5, 10.1, H—C (11)), 3.59-3.49 (br. m, H—C (3)), 2.39-2.12 (m, 2H—C (5)), 1.65-1.38 (m, 2H—C (2)), 2H—C (4), OH), 0.94 (t, J=7.5, MeC(2)).

¹³C-NMR (100 MHz, CDCl₃): δ 137.07 (d), 135.25 (d), 133.31 (d), 131.33 (d), 130.51 (d), 116.44 (t, C(11)), 72.69 (d, C(3)), 36.28 (t, C(5)), 30.20 (t, C(4)), 29.06 (t, C(2)), 9.83 (q, C(1))

MS (EI): 166 (14), 151 (1), 148 (1), 137 (3), 133 (2), 119 (22), 117 (4), 109 (7), 106 (13), 105 (51), 95 (7), 94 (38), 93 (28), 92 (24), 91 (100), 85 (6), 80 (12), 79 (61), 78 (27), 77 (49), 72 (1), 67 (19), 66 (10), 65 (16), 59 (16), 57 (22), 55 (13), 53 (10), 51 (7), 43 (7), 41 (32), 39 (16), 29 (8), 27 (7).

Data of (6Z,8E)-undeca-6,8,10-trien-3-ol:

¹H-NMR (400 MHz, CDCl₃): δ 6.53 (br. dd, J=11.4, 14.9, H—C (8)), 6.41 (dt, J=10.4, 16.9, H—C (10)), 6.21 (br. dd, J=10.7, 14.9, H—C (9)), 6.04 (br. t, J=11.0, H—C (7)), 5.49 (br. dt, J=7.8, 10.8, H—C (6)), 5.22 (br. d, J=16.9, H—C (11)), 5.09 (br. d, J=10.1, H—C(11)), 3.60-3.51 (br. m, H—C (3)), 2.37-2.28 (m, 2H—C (5)), 1.60-1.41 (m, 2H—C (2)), 2H—C(4)), 1.26 (br. s, OH), 0.94 (t, J=7.5, MeC(2)).

¹³C-NMR (100 MHz, CDCl₃): δ 137.14 (d), 133.35 (d), 132.65 (d), 128.79 (d), 128.35 (d), 117.11 (t, C(11)), 72.73 (d, C(3)), 36.58 (t, C(5)), 30.26 (t, C(4)), 24.20 (t, C(2)), 9.85 (q, C(1))

MS (EI): 166 (12), 151 (1), 148 (2), 137 (3), 133 (2), 119 (22), 117 (4), 109 (7), 106 (13), 105 (51), 95 (9), 94 (41), 93 (28), 92 (24), 91 (100), 85 (7), 80 (14), 79 (66), 78 (27), 77 (51), 72 (1), 67 (22), 66 (11), 65 (18), 59 (18), 57 (27), 55 (15), 53 (11), 51 (8), 43 (9), 41 (36), 39 (18), 29 (10), 27 (9).

### EXAMPLE 8

### (4E,6E)-undeca-1,4,6-trien-3-ol

At 20° C., a solution of trans-trans-2,4-nonadienal (8.6 g, 62.4 mmol) in tetrahydrofuran (100 ml) was treated within 45 min. with a 1M solution of vinylmagnesium bromide in tetrahydrofuran (47 ml, 47 mmol). The resulting solution was stirred 2 h at 20° C. before being poured into an ice-cold solution of saturated aqueous NH₄Cl (300 ml). The mixture was extracted twice with methyl tert.-butyl ether (150 ml) and the combined organic phases were washed with saturated aqueous NaCl solution (200 ml), dried (MgSO₄), filtered, and concentrated. FC (hexane/methyl tert.-butyl ether 5:1) of the crude product (11.2 g) gave (4E,6E)-undeca-1,4,6-trien-3-ol (3 g, 38%). Boiling point: 95° C. (0.09 mbar).

Odour description: green, fruity, smoked, ethery

¹H-NMR (400 MHz, CDCl₃): δ 6.21 (br. dd, J=10.4, 15.3, H—C (5)), 6.03 (ddtd, J=0.4, 1.4, 10.4, 15.1, H—C (6)), 5.90 (ddd, J=5.8, 10.4, 17.2, H—C (2)), 5.72 (dt, J=7.0, 14.4, H—C (7)), 5.59 (br. dd, J=6.6, 15.2, H—C (4)), 5.26 (dt., J=1.4, 17.2, H—C (1)), 5.13 (dt., J=1.4, 10.4, H—C (1)), 4.64 (br. d, J=6.2, H—C (3)), 2.08 (br. q, J=7.1, H—C (8)), 1.83 (br. s, OH), 1.41-1.26 (m, 4H), 0.89 (t, J=7.2, Me-C(10)).

¹³C-NMR (100 MHz, CDCl₃): δ 139.41 (d, C(2)), 136.13 (d, C(7)), 131.57 (d, C(5)), 131.20 (d, C(4)), 129.29 (d, C(6)), 114.96 (t, C(1)), 73.60 (d, C(3)), 32.28 (t, C(8)), 31.31 (t, C(9)), 22.18 (t, C(10)), 13.88 (q, MeC(10)).

MS (EI): 166 (3), 148 (10), 137 (2), 133 (1), 123 (6), 119 (7), 109 (35), 105 (19), 96 (28), 95 (32), 91 (44), 85 (7), 83 (14), 82 (13), 81 (50), 79 (43), 77 (28), 67 (34), 65 (14), 57 (16), 55 (100), 54 (20), 53 (19), 43 (14), 41 (45), 39 (24), 29 (12), 27 (17).

UV (CHCl₃): λ (log ε) 282 (2.5), 237 (4.4).

### EXAMPLE 9

### (5E,7E)-dodeca-2,5,7-trien-4-ol

A solution of 1-bromo-1-propene (4.3 ml, 50.6 mmol) in tetrahydrofuran (30 ml) was added to magnesium powder (1.23 g, 50.6 mmol). The reaction mixture was stirred for 2 h at reflux temperature, cooled to 20° C., and treated with a solution of trans-trans-2,4-nonadienal (5.55 g, 36.2 mmol) in tetrahydrofuran (30 ml). The resulting mixture was then stirred for 1 h at 20° C. before being poured into an ice-cold solution of saturated aqueous NH₄Cl (200 ml). The mixture was extracted twice with methyl tert.-butyl ether (100 ml) and the combined organic phases were washed with saturated aqueous NaCl solution (100 ml), dried (MgSO₄), filtered, and concentrated. FC (hexane/methyl tert.-butyl ether 5:1) of the crude product (7.1 g) gave a 86:14 mixture of (2Z,5E,7E)- and (2E,5E,7E)-dodeca-2,5,7-trien-4-ol (3.9 g, 60%). Boiling point: 115° C. (0.08 mbar).

Odour description: fruity, fatty, floral, allylic, metallic

Data of (2Z,5E,7E)-dodeca-2,5,7-trien-4-ol:

¹H-NMR (400 MHz, CDCl₃): δ 6.20 (br. dd, J=10.5, 15.4, H—C (6)), 6.03 (ddtd, J=0.4, 1.4, 10.5, 15.1, H—C (7)), 5.71 (dt, J=7.2, 14.9, H—C (8)), 5.60 (br. dd, J=6.6, 15.4, H—C(5)), 5.60-5.54 (m, H—C (2)), 5.46 (ddq, J=1.8, 8.4, 10.9, H—C (3)), 4.99 (br. t, J=7.5, H—C (4)), 2.08 (br. q, J=7.2, H—C (9)), 1.78 (br. s, OH), 1.69 (dd, J=1.7, 7.0, Me-C(2)), 1.41-1.26 (m, 4H), 0.89 (t, J=7.1, Me-C(11)).

¹³C-NMR (100 MHz, CDCl₃): δ 135.81 (d), 131.72 (d), 131.63 (d), 130.74 (d), 129.41 (d), 126.31 (d), 68.29 (d, C(4)), 32.28 (t), 31.33 (t), 22.18 (t), 13.87 (q), 13.25 (q). Signals of 2Z-isomer δ 135.79 (d), 132.49 (d), 131.94 (d), 130.98 (d), 129.39 (d), 127.22 (d), 73.44 (d, C(4)), 32.31 (t), 31.73 (t), 27.46 (t), 17.68 (q), 13.93 (q).

MS (EI): 180 (8), 162 (28), 137 (4), 123 (23), 119 (24), 109 (11), 105 (52), 95 (35), 93 (22), 91 (89), 85 (13), 84 (24), 81 (33), 79 (53), 77 (34), 69 (100), 67 (33), 65 (19), 57 (17), 55 (56), 53 (20), 43 (26), 41 (55), 39 (23), 29 (9), 27 (11).

Data of (2E,5E,7E)-dodeca-2,5,7-trien-4-ol:

MS (EI): 180 (25), 162 (28), 137 (4), 123 (14), 119 (23), 109 (8), 105 (51), 95 (27), 93 (26), 91 (94), 85 (14), 84 (6), 81 (46), 79 (100), 77 (39), 69 (9), 67 (49), 65 (20), 57 (20), 55 (40), 53 (16), 43 (86), 41 (46), 39 (19), 29 (9), 27 (10).

### EXAMPLE 10

### (5E,7E)-dodeca-1,5,7-trien-4-ol

At 0° C., a solution of trans-trans-2,4-nonadienal (5.55 g, 36.2 mmol) in diethyl ether (50 ml) was treated with a 1M solution of allylmagnesium bromide in diethyl ether (43.4 ml, 43.4 mmol). The resulting solution was stirred 2 h at 20° C. before being poured into an ice-cold solution of saturated aqueous NH₄Cl (200 ml). The mixture was extracted twice with diethyl ether (100 ml) and the combined organic phases were washed with saturated aqueous NaCl solution (100 ml), dried (MgSO₄), filtered, and concentrated. FC (hexane/methyl tert.-butyl ether 5:1) of the crude product (7 g) gave (5E,7E)-undeca-1,5,7-trien-4-ol (3.7 g, 57%). Boiling point: 110° C. (0.1 mbar).

¹H-NMR (400 MHz, CDCl₃): δ 6.20 (br. dd, J=10.4, 15.2, H—C (6)), 6.02 (ddtd, J=0.5, 1.4, 10.4, 15.2, H—C (7)), 5.81 (ddt, J=7.1, 10.4, 17.2, H—C (2)), 5.69 (dt, J=7.1, 15.1, H—C (8)), 5.59 (dd, J=6.6, 15.2, H—C (5)), 5.17-5.10 (m, 2H—C (1)), 4.19 (br. q, J=6.4, H—C(4)), 2.39-2.25 (m, 2H—C (3)), 2.10 (br. q, J=7.0, 2H—C (9)), 1.87 (br. s, OH), 1.42-1.26 (m, 4H), 0.89 (t, J=7.2, Me).

¹³C-NMR (100 MHz, CDCl₃): δ 135.67 (d), 134.23 (d), 132.47 (d), 131.08 (d), 129.32 (d), 118.11 (t, C(1)), 71.53 (d, C(4)), 41.92 (t), 32.28 (t), 31.33 (t), 22.20 (t), 13.88 (q).

MS (EI): 180 (1), 162 (8), 140 (9), 139 (95), 137 (1), 121 (11), 119 (5), 109 (2), 105 (16), 97 (13), 96 (7), 95 (21), 93 (24), 91 (32), 83 (100), 81 (26), 79 (45), 77 (18), 71 (8), 69 (25), 67 (38), 65 (13), 57 (24), 55 (71), 53 (16), 43 (24), 41 (51), 39 (21), 29 (8), 27 (9).

Odour description: fatty, fruity, floral, animalic, green, allylic

### EXAMPLE 11

### 7-, 9-, 10-hydroxy-1,3,5-undecatriene

The enzymatic reaction was conducted at 37° C. using 2.5 ml of an enzyme mixture (BD Biosciences) containing: 5 nmole of CYP2E1; protein content of 6.3 mg/ml in 100 mM potassium phosphate (pH 7.4); cytochrome c reductase activity corresponding to 2000 nmole/(min×mg protein); cytochrome b5 content of 280 pmol per mg protein. The total volume of the reaction was 50 ml.

The initial mixture was prepared using 43 ml water, 2.5 ml 1 M potassium phosphate and 2.5 ml enzyme mixture. A first portion of the substrate 1,3,5-undecatriene was added: 20 mg of a 20% solution of undecatriene in acetone. The reaction mixture was incubated for 10 min at 37° C. (waterbath) in an Erlenmeyer flask which was loosely capped with aluminum foil. To initiate enzymatic catalysis, 2 ml of 50 mM NADPH was added to the reaction mixture (2 mM final concentration). The incubation at 37° C. took place for 4 hours, and at 30, 60, 90, 120, 150, 180 and 210 minutes, additional portions of 10 mg of a 20% solution of undecatriene in acetone were added. Following the reaction, the mixture was chilled on ice and insoluble materials separated by centrifugation. The supernatant containing substrate and products was diluted with one volume water, and the compounds were isolated using a solid-phase-extraction column to remove water and salts. The mixture was loaded during 1.5 hours onto an ISOLUTE ENV+solid-phase-extraction (SPE) column (Biotage Sweden AB, Kungsgatan 76, SE-753 18 Uppsala, Sweden) which consists of a hydroxylated polystyrene-divinylbenzene copolymer. Stepwise elution was done using acetone (5×0.5 ml) and the different fractions analyzed by GC and GC-MS to identify potential products. Using preparative GC, the produced metabolites were isolated and their structure elucidated using GC-MS and NMR analysis.

Four different metabolites have been detected: 7-hydroxy-1,3,5-undecatriene, 9-hydroxy-1,3,5-undecatriene and 10-hydroxy-1,3,5-undecatriene. For 7-hydroxy-1,3,5-undecatriene, the two isomers 7-hydroxy-(E,E)-1,3,5-undecatriene and 7-hydroxy-(E,Z)-1,3,5-undecatriene have been identified.

### EXAMPLE 12

### A Green-Marine Composition for Toiletries

| Compound/Ingredient | parts by weight 1/1000 |
|---|---|
| Acetal CD (phenylacetaldehyde glycerylacetal) | 40 |
| Acetal R (acetaldehyde phenylethyl propyl acetal) | 2 |
| Adoxal 10% DPG (2,6,10-trimethyl-9-undecenal) | 1.5 |
| Phenoxyethyl alcohol | 70 |
| Lynalyl benzoate | 100 |
| Bigarade oil | 10 |
| Clonal (10% in DPG) | 5 |
| Beta-damascone | 5 |
| Dimetol (2,6-dimethyl-2-heptanol) | 25 |
| Disopropylene Glycol | 375 |
| Florhydral | 20 |
| Glycolierral | 60 |
| (Z)-Hex-3-en-1-ol | 5 |
| (Z)-Hex-3-en-1-yl acetate | 2.5 |
| (Z)-Hex-3-en-1-yl formiate | 5 |
| (Z)-Hex-3-en-1-yl salicylate | 5 |
| Hexyl propionate | 10 |
| Beta-ionone | 10 |
| Linalool | 100 |
| Linalool oxide (1% DPG) | 2.5 |
| Melonal (10% DPG) | 2.5 |
| Menthe Crepue Ess USA (10% DPG) | 10 |
| Nerol | 80 |
| Nerolidol | 40 |
| Radjanol | 5 |
| Viridine (10% PE) | 5 |
| Undeca-6,8,10-trien-5-ol of Example 2 | 4 |

The undeca-6,8,10-trien-5-ol of Example 2 brings a fresh fruity galbanum note to the composition. and enhances its volume.

1. A flavor or fragrance compound of formula (I) 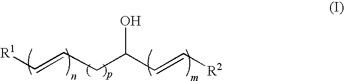
wherein
p is 0 or an integer of 1 to 7;
n is 1, 2, or 3; m is 0, 1 or 2; and n+m=3;
R¹ is hydrogen, or linear C₁-C₆ alkyl;
R² is hydrogen, or linear C₁-C₆ alkyl; and
the total number of carbon atoms of the compound is 7 to 16;
with the proviso that if m is 0, R² is linear C₁-C₆ alkyl.

2. A flavor or fragrance compound according to claim 1 wherein the compound is selected from:
(6E,8E)-undeca-6,8,10-trien-5-ol,
(5E,7E,9E)-undeca-5,7,9-trien-4-ol,
(5E,7E,9Z)-undeca-5,7,9-trien-4-ol,
(6Z,8E)-undeca-6,8,10-trien-5-ol,
(6E,8E)-undeca-6,8,10-trien-5-ol,
(6E,8E)-undeca-6,8,10-trien-2-ol,
(6E,8E)-undeca-6,8,10-trien-3-ol,
(4E,6E)-undeca-1,4,6-trien-3-ol,
(5E,7E)-dodeca-2,5,7-trien-4-ol and
(5E,7E)-dodeca-1,5,7-trien-4-ol.

3. A flavor or fragrance composition comprising a compound of formula (I) according to claim 1.

4. A flavour or fragrance application comprising a compound according to of formula (I) according to in claim 1, and a consumer product base.

5. A fragrance application according to claim 4 wherein the fragrance application is selected from: fine fragrance products, household products, laundry products, body care products, and cosmetics.

6. A method of improving, enhancing or modifying a flavor or fragrance application comprising the step of: adding to the flavor or fragrance composition a compound according to formula (I) as defined in claim 1.

7. A method of producing a compound of formula (I) as defined in claim 1, comprising the step of:
contacting a compound of formula (II) 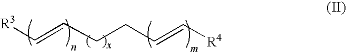
wherein
x is 0 or an integer of 1 to 7;
n is 1, 2, or 3; m is 0, 1 or 2; and n+m=3;
R³ is hydrogen, or linear C₁-C₆ alkyl;
R⁴ is hydrogen, or linear C₁-C₆ alkyl; and
the total number of carbon atoms of the compound is 7 to 16;
with the proviso that if m is 0, R⁴ is linear C₁-C₆ alkyl;
with a cytochrome P450 enzyme.

8. A method according to claim 7 wherein the cytochrome P450 enzyme is CYP2E1.

9. A compound of formula (I) selected from the group consisting of:
(6E,8E)-undeca-6,8,10-trien-5-ol,
(5E,7E,9E)-undeca-5,7,9-trien-4-ol,
(5E,7E,9Z)-undeca-5,7,9-trien-4-ol,
(6Z,8E)-undeca-6,8,10-trien-5-ol,
(6E,8E)-undeca-6,8,10-trien-5-ol,
(6E,8E)-undeca-6,8,10-trien-2-ol,
(6E,8E)-undeca-6,8,10-trien-3-ol,
(5E,7E)-dodeca-2,5,7-trien-4-ol and
(5E,7E)-dodeca-1,5,7-trien-4-ol.
